SEQUENCE LISTING (25 sequences)

SEQ ID NO: 1 - protein, 46 aa, synthetic construct
LAEAKVLANRELDKYGVSDFYKRLINKAKTVEGVEALKLHILAALP

SEQ ID NO: 2 - protein, 106 aa, Squalus acanthias
TRVDQSPSSLSASVGDRVTITCVLTDTSYPLYSTYWYQQKPGSSNKEQISISGRYSESVNKGTKSFTLTISSLQPEDFATYYCRAMGTNIWTGDGAGTKVEIKAAA

SEQ ID NO: 3 - protein, 16 aa, synthetic construct
QRFCTGHFGGLHPCNG

SEQ ID NO: 4 - protein, 249 aa, synthetic construct
MGVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKHHHHHH

SEQ ID NO: 5 - protein, 247 aa, synthetic construct
MGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIHHHHHH

SEQ ID NO: 6 - protein, 246 aa, synthetic construct
MGQVQLKESGPGLVAPSQSLSITCTVSGFSVTNYGVHWVRQPPGKGLEWLGVIWAGGITNYNSAFMSRLSISKDNSKSQVFLKMNSLQIDDTAMYYCASRGGHYGYALDYWGQGTSVTVSSGGGGSGGGGSGGGGSSIVMTQTPKFLLVSAGDRVTITCKASQSVSNDVTWYQQKAGQSPKLLIYSASNRYSGVPDRFTGSGYGTAFTFTISTVQAEDLAVYFCQQDYSSFGGGTKLEIKHHHHHH

SEQ ID NO: 7-8 - skipped (2 entries)

SEQ ID NO: 9 - protein, 5 aa, synthetic construct
GGGGS

SEQ ID NO: 10 - protein, 13 aa, synthetic construct
GGGGSGGSGGGGS

SEQ ID NO: 11 - protein, 22 aa, synthetic construct
GGGGSGGSGGSGGSGGSGGGGS

SEQ ID NO: 12 - protein, 15 aa, synthetic construct
GGGGSGGGGSGGGGS

SEQ ID NO: 13 - protein, 297 aa, synthetic construct
MGVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGTSLAEAKVLANRELDKYGVSDFYKRLINKAKTVEGVEALKLHILAALPTSGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKHHHHHH

SEQ ID NO: 14 - protein, 306 aa, synthetic construct
MGVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGTSGSGGSLAEAKVLANRELDKYGVSDFYKRLINKAKTVEGVEALKLHILAALPGGSGTSGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKHHHHHH

SEQ ID NO: 15 - protein, 294 aa, synthetic construct
MGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSLAEAKVLANRELDKYGVSDFYKRLINKAKTVEGVEALKLHILAALPGGSGTSGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKHHHHHH

SEQ ID NO: 16 - protein, 297 aa, synthetic construct
MGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGTSGSGGSLAEAKVLANRELDKYGVSDFYKRLINKAKTVEGVEALKLHILAALPGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKHHHHHH

SEQ ID NO: 17 - protein, 299 aa, synthetic construct
MGLAEAKVLANRELDKYGVSDFYKRLINKAKTVEGVEALKLHILAALPGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKHHHHHH

SEQ ID NO: 18 - protein, 295 aa, synthetic construct
MGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGTSLAEAKVLANRELDKYGVSDFYKRLINKAKTVEGVEALKLHILAALPTSGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIHHHHHH

SEQ ID NO: 19 - protein, 294 aa, synthetic construct
MGQVQLKESGPGLVAPSQSLSITCTVSGFSVTNYGVHWVRQPPGKGLEWLGVIWAGGITNYNSAFMSRLSISKDNSKSQVFLKMNSLQIDDTAMYYCASRGGHYGYALDYWGQGTSVTVSSGGGGSGTSLAEAKVLANRELDKYGVSDFYKRLINKAKTVEGVEALKLHILAALPTSGSGGGGSSIVMTQTPKFLLVSAGDRVTITCKASQSVSNDVTWYQQKAGQSPKLLIYSASNRYSGVPDRFTGSGYGTAFTFTISTVQAEDLAVYFCQQDYSSFGGGTKLEIKHHHHHH

SEQ ID NO: 20 - protein, 354 aa, synthetic construct
MGQVQLKESGPGLVAPSQSLSITCTVSGFSVTNYGVHWVRQPPGKGLEWLGVIWAGGITNYNSAFMSRLSISKDNSKSQVFLKMNSLQIDDTAMYYCASRGGHYGYALDYWGQGTSVTVSSGGGGSGTSTRVDQSPSSLSASVGDRVTITCVLTDTSYPLYSTYWYQQKPGSSNKEQISISGRYSESVNKGTKSFTLTISSLQPEDFATYYCRAMGTNIWTGDGAGTKVEIKAAATSGSGGGGSSIVMTQTPKFLLVSAGDRVTITCKASQSVSNDVTWYQQKAGQSPKLLIYSASNRYSGVPDRFTGSGYGTAFTFTISTVQAEDLAVYFCQQDYSSFGGGTKLEIKHHHHHH

SEQ ID NO: 21 - protein, 264 aa, synthetic construct
MGQVQLKESGPGLVAPSQSLSITCTVSGFSVTNYGVHWVRQPPGKGLEWLGVIWAGGITNYNSAFMSRLSISKDNSKSQVFLKMNSLQIDDTAMYYCASRGGHYGYALDYWGQGTSVTVSSGGGGSGTSQRFCTGHFGGLHPCNGTSGSGGGGSSIVMTQTPKFLLVSAGDRVTITCKASQSVSNDVTWYQQKAGQSPKLLIYSASNRYSGVPDRFTGSGYGTAFTFTISTVQAEDLAVYFCQQDYSSFGGGTKLEIKHHHHHH

SEQ ID NO: 22 - protein, 302 aa, synthetic construct
MGEVQLVESGGGLVQPGGSLRLSCAASGFNIKDTYIHWVRQAPGKGLEWVARIYPTNGYTRYAGGSLAEAKVLANRELDKYGVSDFYKRLINKAKTVEGVEALKLHILAALPGGSDSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKHHHHHH

SEQ ID NO: 23 - protein, 302 aa, synthetic construct
MGEVQLVESGGGLVQPGGSLGGSLAEAKVLANRELDKYGVSDFYKRLINKAKTVEGVEALKLHILAALPGGSRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKHHHHHH

SEQ ID NO: 24 - protein, 302 aa, synthetic construct
MGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGGGSLAEAKVLANRELDKYGVSDFYKRLINKAKTVEGVEALKLHILAALPGGSVPSRFSGSRSGTDFTLTISSLQPEDFATYYCQQHYTTPPTFGQGTKVEIKHHHHHH

SEQ ID NO: 25 - protein, 302 aa, synthetic construct
MGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITGGSLAEAKVLANRELDKYGVSDFYKRLINKAKTVEGVEALKLHILAALPGGSCRASQDVNTAVAWYQQKPGKAPKLLIYSASFLYSGVPSRFSGSRSGTDFTLTISSLQPEDFATYYCQQHYTTPPTFGQGTKVEIKHHHHHH

(19) United States
(12) Patent Application Publication
(10) Pub. No.: US 2026/0327335 A1
(43) Pub. Date: Oct. 22, 2026

(54) MODIFIED PROTEIN OF SINGLE CHAIN VARIABLE FRAGMENT

(71) Applicant: ProAbtech Co., Ltd., Gwangju (KR)

(72) Inventors: In Chan KWON, Gwangju (KR); Na Hyun KWON, Gwangju (KR); Jae-Hun LEE, Gwangju (KR)

(21) Appl. No.: 19/104,534

(22) PCT Filed: Aug. 21, 2023
(86) PCT No.: PCT/KR2023/012335

§ 371 Date: Feb. 18, 2025

(30) Foreign Application Priority Data
Aug. 19, 2022 (KR) 10-2022-0103901
Aug. 21, 2023 (KR) 10-2023-0108880

(51) Int. Cl. C07K 16/32

(52) U.S. Cl. CPC C07K 16/32; C07K 2317/622; C07K 2317/92; C07K 2319/70

(57) ABSTRACT

The present invention provides a modified protein of a single chain variable (scFv) fragment having an albumin binding moiety fused to a single chain variable fragment in which a heavy chain variable domain (V_{H}) and a light chain variable domain (V_{L}) are connected by a linker.

### CROSS-REFERENCE TO RELATED APPLICATIONS

This application is a national phase application of PCT Application No. PCT/KR2023/012335, filed on 21 Aug. 2023, which claims priority to Korean Patent Application Nos. 10-2022-0103901, filed on 19 Aug. 2022 and 10-2023-0108880, filed on 21 Aug. 2023. The entire disclosures of the applications identified in this paragraph are incorporated herein by references.

### SEQUENCE LISTING

This application contains references to amino acid sequences and/or nucleic acid sequences which have been submitted concurrently herewith as the sequence listing XML file entitled “000100usnp_SequenceListing.XML”, file size 26,028 bytes, created on 18 Feb. 2025. The aforementioned sequence listing is hereby incorporated by reference in its entirety pursuant to 37 C.F.R. § 1.52(e)(5).

### TECHNICAL FIELD

The present disclosure relates to a modified protein of single-chain variable fragment (scFv), which is utilizable in medical fields.

### BACKGROUND ART

Therapeutic antibodies were approved for use in 1986, and the market thereof has grown rapidly since then. Existing monoclonal antibodies are expensive, require laborious mammalian expression systems, and have tissue penetration issues. Due to these problems, research has been conducted on various antibody fragment formats as alternatives.

Among all these antibody derivatives, scFvs demonstrated clinical applicability as several scFv-based therapeutics were approved by the FDA in 1988. An scFv is a structure in which a heavy chain variable domain (V_{H}) and a light chain variable domain (V_{L}) of immunoglobulins (Igs) are linked by a polypeptide linker. Such scFvs do not cause unwanted crystallizable fragment (Fc)-mediated immune responses and are more effective in tumor penetration and distribution than full-length antibodies. However, despite these advantages, there is a disadvantage of extremely short half-life because the scFvs in serum are typically small in size (about 25 kDa) and lack Fc regions.

Accordingly, the clinical application of scFvs is limited to a few cases, such as hematologic malignancy and macular degeneration, despite their usefulness. Thus, there is a need to develop new scFv-based therapeutics that can address this circumstance.

### DISCLOSURE

### Technical Problem

The present disclosure aims to provide a modified protein of scFv with a novel structure.

The present disclosure aims to provide a pharmaceutical composition for the prevention or treatment of cancer, comprising the modified protein of scFv.

### Technical Solution

1. A single chain variable fragment modified protein, which is a form where an albumin binding moiety is fused to a single chain variable fragment (scFv) in which a heavy chain variable domain(VH) and a light chain variable domain(VL) of an antibody are linked by a linker.

2. The single chain variable fragment modified protein of above 1, wherein the albumin binding moiety is fused within the linker or at the N-terminal of the scFv or at the C-terminal of the scFv.

3. The single chain variable fragment modified protein of above 1, wherein the albumin binding moiety is fused within the linker, and wherein a bioactive substance is fused at the C-terminal of the scFv.

4. The single chain variable fragment modified protein of above 1, wherein the albumin binding moiety is fused at a position separated by at least 3 aa from the C-terminal of the heavy chain variable domain(VH) and the N-terminal of the light chain variable domain(VL), respectively

5. The single chain variable fragment modified protein of above 1, wherein the albumin binding moiety is positioned in the opposite direction from the direction of the complementary determining region (CDR) of the heavy chain variable domain(VH) and the light chain variable domain(VL).

6. The single chain variable fragment modified protein of above 1, wherein the length of the albumin binding moiety is 10 to 150 aa.

7. The single chain variable fragment modified protein of above 1, wherein the length of the linker is 6 to 50 aa.

8. The single chain variable fragment modified protein of above 1, wherein the length of the scFv is 150 to 350 aa.

9. The single chain variable fragment modified protein of above 1, wherein the length of the heavy chain variable domain(V_{H}) and the light chain variable domain(V_{L}) is 70 to 150 aa, respectively.

10. The single chain variable fragment modified protein of above 1, wherein the albumin binding moiety comprises an amino acid sequence selected from a sequence of SEQ ID NO. 1 to 3.

11. The single chain variable fragment modified protein of above 1, wherein the scFv comprises an amino acid sequence selected from a sequence of SEQ ID NO. 4 to 6.

12. The single chain variable fragment modified protein of above 1, wherein the linker comprises an amino acid sequence of (GS)n, (GGS)n or (GGGGS)n.

13. The single chain variable fragment modified protein of above 3, wherein the bioactive substance is selected from the group consisting of cytokine, insulin, insulin-like growth factor (IGF)-1, IGF-2, epidermal growth factor (EGF), transforming growth factor (TGF), human growth hormone, and vascular endothelial growth factor (VEGF).

14. A pharmaceutical composition for the treatment or prevention of cancer comprises a single chain variable fragment modified protein of any of above 1-13.

### Advantageous Effects

A scFv modified protein of the present disclosure exhibits excellent binding capacity to an antigen because the albumin-binding site does not interfere with the complementary determining region (CDR) of a heavy chain variable region and a light chain variable region.

The scFv modified protein of the present disclosure binds to albumin present in the body, thus having an extended half-life.

The scFv modified protein of the present disclosure can appropriately adjust the length of a linker and an albumin-binding moiety, thus maintaining excellent binding capacity to both an antibody and albumin.

The scFv modified protein of the present disclosure can deliver a bioactive substance such as cytokine to a target by conjugating it to the C-terminal.

The scFv modified protein of the present disclosure has excellent cell penetration ability.

The scFv modified protein of the present disclosure can be easily prepared using a microbial production system.

The scFv modified protein of the present disclosure can fuse the albumin-binding moiety while minimizing structural changes in known scFvs and thus can be utilized appropriately in the improvement of the antibody and fragments thereof.

### DESCRIPTION OF DRAWINGS

FIGS. 1, 2, 3, 4, 5, 6, 7, 8, 9, 10, 11, 12, 13 and 14 illustrate 4D5scFv, examples and comparative examples of the present application, and structures thereof implemented by AlphaFold2.

FIG. 15 shows computational evaluation results of structure predictions for 4D5scFv and 4D5-ABD variants. (a) model structures predicted by AlphaFold2 and visualized by PyMOL. (b) per-position pLDDT score for structure prediction as reported by AlphaFold2.

FIG. 16 shows 4D5scFv and 4D5-ABD variants aligned and visualized by PyMOL.

FIG. 17 shows complex conformational structures of two 4D5-ABD variants and HER2 (PDB ID:1N8Z) visualized by PyMOL.

FIGS. 18a and 18b show sodium dodecyl sulfate polyacrylamide gel electrophoresis (SDS-PAGE) analysis results for expression and purification of 4D5scFv and 4D5-ABD variants. (a) Coomassie blue-stained SDS-PAGE gels of cell lysate samples achieved during expression (MW: molecular weight standard; BI: cell lysate samples before induction; AI: cell lysate samples after induction). (b) Coomassie blue-stained SDS-PAGE gels of purified samples.

FIG. 19 shows SDS-PAGE analysis results of 4D5scFv and two 4D5-ABD variants under reducing (left) and non-reducing (right) conditions (MW: molecular weight standard).

FIG. 20 shows MALDI-TOF analysis results of purified 4D5scFv and 4D5-ABD variants.

FIG. 21 shows size exclusion chromatogram of 4D5scFv, 4D5-ABD variants, and standard proteins (4D5scFv: 27 kDa; 4D5scFv dimer: 54 kDa; 4D5-S-ABD: 32 kDa; 4D5-L-ABD: 33 kDa; ovalbumin (OA): 43 kDa; conalbumin (CA): 75 kDa; aldolase (Ald): 158 kDa).

FIG. 22 shows anti-HER2 enzyme-linked immunosorbent assay (ELISA) results of 4D5scFv and 4D5-ABD variants under conditions in the presence and absence of HSA.

FIG. 23 shows anti-HSA ELISA results of 4D5scFv and 4D5-ABD variants.

FIG. 24 shows a pharmacokinetic profile of 4D5scFv and 4D5-ABD variants.

### MODE FOR INVENTION

An antibody, which may be selected appropriately depending on the usage of a modified protein of the present disclosure, is not limited to particular types.

For example, this antibody may be an antibody against a disease antigen, and this disease may be an autoimmune disease or cancer.

The antibody may be an anticancer drug or an immunosuppressive drug. Specifically, the antibody may be selected from the group consisting of trastuzumab, naxitamab, cetuximab, telisotuzumab, tisotumab, pinatuzumab, lifastuzumab, indusatumab, vandortuzumab, sofituzumab, vorsetuzumab, trastuzumab, mirvetuximab, coltuximab, naratuximab, indatuximab, anetumab, lorvotuzumab, cantuzumab, laprituximab, bivatuzumab, vadastuximab, rovalpituzumab, inotuzumab, sacituzumab, labetuzumab, milatuzumab, lupartumab, aprutumab, pembrolizumab, atezolizumab, and OKT3 (Anti-CD3 monoclonal antibody), but there are no limitations.

An scFv is not limited to those having a specific amino acid length as long as it can fuse an albumin-binding moiety depending on the purpose of the present disclosure.

The amino acid length of the scFv may, for example, be at least 100 aa, at least 110 aa, at least 120 aa, at least 130 aa, at least 140 aa, at least 150 aa, at least 160 aa, at least 170 aa, at least 180 aa, at least 190 aa, at least 200 aa, at least 210 aa, at least 220 aa, at least 230 aa, at least 240 aa, at least 250 aa, at least 260 aa, at least 270 aa, at least 280 aa, at least 290 aa, at least 300 aa, at least 310 aa, at least 320 aa, at least 330 aa, at least 340 aa, at least 350 aa, at least 360 aa, at least 370 aa, at least 380 aa, at least 390 aa, at least 400 aa, at least 410 aa, at least 420 aa, at least 430 aa, at least 440 aa, at least 450 aa, at least 460 aa, at least 470 aa, at least 480 aa, at least 490 aa, or at least 500 aa.

The amino acid length of the scFv may, for example, be not more than 500 aa, not more than 490 aa, not more than 480 aa, not more than 470 aa, not more than 460 aa, not more than 450 aa, not more than 440 aa, not more than 430 aa, not more than 420 aa, not more than 410 aa, not more than 400 aa, not more than 390 aa, not more than 380 aa, not more than 370 aa, not more than 360 aa, not more than 350 aa, not more than 340 aa, not more than 330 aa, not more than 320 aa, not more than 310 aa, not more than 300 aa, not more than 290 aa, not more than 280 aa, not more than 270 aa, not more than 260 aa, not more than 250 aa, not more than 240 aa, not more than 230 aa, not more than 220 aa, not more than 210 aa, not more than 200 aa, not more than 190 aa, not more than 180 aa, not more than 170 aa, not more than 160 aa, not more than 150 aa, not more than 140 aa, not more than 130 aa, not more than 120 aa, not more than 110 aa, or not more than 100 aa.

The scFv may have a length of, for example, 150 to 350 aa, 160 to 340 aa, 170 to 330 aa, 180 to 320 aa, 190 to 310 aa, or 200 to 300 aa.

A heavy chain variable domain(V_{H}) and a light chain variable domain (V_{L}) are not limited to those having a specific amino acid length.

The amino acid length of the heavy chain variable domain(V_{H}) and the light chain variable domain(V_{L}) may, for example, be at least 50 aa, at least 55 aa, at least 60 aa, at least 65 aa, at least 70 aa, at least 75 aa, at least 80 aa, at least 85 aa, at least 90 aa, at least 95 aa, at least 100 aa, at least 105 aa, at least 110 aa, at least 115 aa, at least 120 aa, at least 125 aa, at least 130 aa, at least 135 aa, at least 140 aa, at least 145 aa, at least 150 aa, at least 155 aa, at least 160 aa, at least 165 aa, at least 170 aa, at least 175 aa, at least 180 aa, at least 185 aa, at least 190 aa, at least 195 aa, at least 200 aa, at least 205 aa, at least 210 aa, at least 215 aa, at least 220 aa, at least 225 aa, at least 230 aa, at least 235 aa, at least 240 aa, at least 245 aa, or at least 250 aa, respectively.

The amino acid length of the heavy chain variable domain(V_{H}) and the light chain variable domain(V_{L}) may, for example, be not more than 250 aa, not more than 245 aa, not more than 240 aa, not more than 235 aa, not more than 230 aa, not more than 225 aa, not more than 220 aa, not more than 215 aa, not more than 210 aa, not more than 205 aa, not more than 200 aa, not more than 195 aa, not more than 190 aa, not more than 185 aa, not more than 180 aa, not more than 175 aa, not more than 170 aa, not more than 165 aa, not more than 160 aa, not more than 155 aa, not more than 150 aa, not more than 145 aa, not more than 140 aa, not more than 135 aa, not more than 130 aa, not more than 125 aa, not more than 120 aa, not more than 115 aa, not more than 110 aa, not more than 105 aa, not more than 100 aa, not more than 95 aa, not more than 90 aa, not more than 85 aa, not more than 80 aa, not more than 75 aa, not more than 70 aa, not more than 65 aa, not more than 60 aa, not more than 55 aa, or not more than 50 aa, respectively.

The heavy chain variable domain and the light chain variable domain may have a length of, for example, 70 to 150 aa, 80 to 140 aa, 90 to 130 aa, or 100 to 120 aa, respectively.

A linker, which is a peptide linker, allows structural stability to be maintained and binding to an antigen in the correct orientation by linking the heavy chain variable domain and the light chain variable domain and adjusting the distance and orientation.

For example, the amino acid length of the linker may be at least 3 aa, at least 4 aa, at least 5 aa, at least 6 aa, at least 7 aa, at least 8 aa, at least 9 aa, at least 10 aa, at least 11 aa, at least 12 aa, at least 13 aa, at least 14 aa, at least 15 aa, at least 16 aa, at least 17 aa, at least 18 aa, at least 19 aa, at least 20 aa, at least 21 aa, at least 22 aa, at least 23 aa, at least 24 aa, at least 25 aa, at least 26 aa, at least 27 aa, at least 28 aa, at least 29 aa, or at least 30 aa.

In addition, the amino acid length of the linker may be not more than 100 aa, not more than 95 aa, not more than 90 aa, not more than 85 aa, not more than 80 aa, not more than 75 aa, not more than 70 aa, not more than 65 aa, not more than 60 aa, not more than 55 aa, not more than 50 aa, not more than 55 aa, not more than 50 aa, not more than 45 aa, not more than 40 aa, not more than 35 aa, not more than 30 aa, not more than 25 aa, not more than 20 aa, not more than 15 aa, or not more than 10 aa.

For example, the linker may have a length of 6 to 50 aa, 6 to 40 aa, 6 to 30 aa, or 6 to 20 aa.

The linker is not limited to a specific sequence as long as it can maintain the flexibility and structural stability of the scFv.

For example, the linker may consist of (GS (SEQ ID NO: 7))ₙ, (GGS (SEQ ID NO: 8))ₙ, (GGGGS (SEQ ID NO: 9))ₙ, or a combination thereof. In this case, n above is a natural number, which is 1 to 10, and may, for example, be 1, 2, 3, 4, 5, 6, 7, 8, 9, or 10. The order and repetition in the combination thereof are not limited, and at least two types may be combined.

The linker may have a sequence of SEQ ID NOs: 10 to 12.

The scFv may further comprise a start restriction sequence (for example, MG) at the N-terminal.

The C-terminal of the scFv may further comprise a His-tag sequence (for example, HHHHHH) to isolate and purify a modified scFv protein.

For example, the scFv may have a sequence of SEQ ID NO: 4 (4D5scFv), SEQ ID NO: 5 (OKTscFv), or SEQ ID NO: 6 (3F8scFv).

As used herein, the term albumin-binding moiety (ABM) refers to a part that interacts with an albumin protein and may, for example, be an antibody, an antibody analog, a protein domain, a protein motif, a peptide, a compound, an aptamer, an oligonucleotide, a saccharide, or the like.

The amino acid length of the ABM may, for example, be at least 5 aa, at least 6 aa, at least 7 aa, at least 8 aa, at least 9 aa, at least 10 aa, at least 11 aa, at least 12 aa, at least 13 aa, at least 14 aa, at least 15 aa, at least 16 aa, at least 17 aa, at least 18 aa, at least 19 aa, at least 20 aa, at least 21 aa, at least 22 aa, at least 23 aa, at least 24 aa, at least 25 aa, at least 26 aa, at least 27 aa, at least 28 aa, at least 29 aa, or at least 30 aa, at least 31 aa, at least 32 aa, at least 33 aa, at least 34 aa, at least 35 aa, at least 36 aa, at least 37 aa, at least 38 aa, at least 39 aa, at least 40 aa, at least 41 aa, at least 42 aa, at least 43 aa, at least 44 aa, at least 45 aa, at least 46 aa, at least 47 aa, at least 48 aa, at least 49 aa, or at least 50 aa.

The amino acid length of the ABM may, for example, be not more than 200 aa, not more than 195 aa, not more than 190 aa, not more than 185 aa, not more than 180 aa, not more than 175 aa, not more than 170 aa, not more than 165 aa, not more than 160 aa, not more than 155 aa, not more than 150 aa, not more than 145 aa, not more than 140 aa, not more than 135 aa, not more than 130 aa, not more than 125 aa, not more than 120 aa, not more than 115 aa, not more than 110 aa, not more than 105 aa, not more than 100 aa, not more than 95 aa, not more than 90 aa, not more than 85 aa, not more than 80 aa, not more than 75 aa, not more than 70 aa, not more than 65 aa, not more than 60 aa, not more than 55 aa, or not more than 50 aa.

The ABM may have a length of, for example, 10 to 150 aa, 30 to 100 aa, or 30 to 50 aa.

For example, the ABM may have a sequence of SEQ ID NO: 1 (ABD), SEQ ID NO: 2 (VNAR (Variable domain of new antigen receptors)), or SEQ ID NO: 3 (ABP (Albumin binding protein)).

The ABM is introduced at a selected position where the structure or antigen-binding function of the scFv is not significantly compromised. The ABM may be fused within the linker or at the N-terminal or C-terminal of the scFv.

Depending on the binding site of the ABM, the scFv may be schematized as (i) ABM-V_{H}-linker-V_{L}, (ii) V_{H}-linker-V_{L}-ABM, (iii) V_{H}-ABM-linker-V_{L}, (iv) V_{H}-linker1-ABM-linker2-V_{L}, (v) V_{H}-linker-ABM-V_{L}, and the like.

When the ABM is fused at the N-terminal or C-terminal as in (i) and (ii), another linker may be added to link the ABM. Examples of such forms may include (vi) ABM-linker1-V_{H}-linker2-V_{L} and (vii) V_{H}-linker1-V_{L}-linker2-ABM.

linker1 and linker2 may differ from each other in amino acid sequence and length.

The amino acid length of linker1 and linker2 may be at least 3 aa, at least 5 aa, at least 7 aa, at least 9 aa, or at least 10 aa, respectively. The upper limit thereof may be not more than 15 aa, not more than 20 aa, not more than 25 aa, not more than 30 aa, or not more than 50 aa, but there are no limitations.

When fused within the linker, the ABM is preferably fused at a position that is each separated by at least 2 aa, at least 3 aa, at least 4 aa, at least 5 aa, at least 6 aa, at least 7 aa, at least 8 aa, at least 9 aa, or at least 10 aa from the C-terminal of the heavy chain variable domain and the N-terminal of the light chain variable domain, respectively.

For example, the ABM may be fused between one of the 4th to 8th amino acids of a linker having a length of 10 to 15 aa and an adjacent amino acid. Alternatively, the ABM may be fused between one of the 7th to 14th amino acids of a linker having a length of 16 to 20 aa and an adjacent amino acid.

Specifically, the ABM may be fused between one of the 5th to 8th amino acids of a linker of SEQ ID NO: 10 or 11 and an adjacent amino acid or may be fused between one of the 11th to 16th amino acids of a linker of SEQ ID NO: 12 and an adjacent amino acid.

Alternatively, the ABM may be fused between the 6th and 7th amino acids of a linker of SEQ ID NO: 10 or 11 or between the 13th and 14th amino acids of a linker of SEQ ID NO: 12, but there are no limitations.

When the ABM is not fused at a position that is separated by at least 3 aa from the C-terminal of the heavy chain variable domain and the N-terminal of the light chain variable domain, interference with adjacent CDRs may occur, resulting in poor binding capacity of the antibody.

When the ABM is fused within the linker or at the C-terminal of the scFv, the ABM is positioned in the opposite direction to the direction in which the CDRs of the heavy chain variable domain and the light chain variable domain are located. In this case, the opposite direction means that, for example, when the CDRs are located on the lower side of the modified scFv protein, the ABM is positioned on the upper side. In addition, the opposite direction means that, for example, when the CDRs are located on the left side of the modified scFv protein, the ABM is positioned on the right side.

Albumin may be human serum albumin (HSA).

The modified protein of the present disclosure may bind to serum albumin via the ABM when administered in vivo, thus delaying the metabolism and degradation of the modified protein and increasing cancer cell permeability.

When the ABM is fused within the linker of the scFv, a bioactive substance may be bound additionally to the N-terminal or C-terminal of the scFv. In addition, when the ABM is fused at a position other than the C-terminal of the scFv, the bioactive substance may be bound to the C-terminal of the scFv. In this case, the modified scFv protein of the present disclosure functions as a carrier to deliver the bioactive substance to a target.

This bioactive substance refers to any material capable of exhibiting a predetermined activity when administered in vivo. The bioactive substance may be any one selected from the group consisting of, for example, cytokines, insulin, Insulin-like Growth Factor-1 (IGF-1), Insulin-like Growth Factor-2 (IGF-2), (Epidermal Growth Factor (EGF), Transforming Growth Factor (TGF), human growth hormone, and Vascular Endothelial Growth Factor (VEGF).

Examples of such cytokines may include interleukins (IL-1 to IL-17), tumor necrosis factor (TNF), interferons (IFNs), erythropoietin (EPO), thrombopoietin (TPO), and the like.

The ABM may be fused at the center of the amino acid sequence of the linker in the scFv. Alternatively, the ABM may be fused between an amino acid distant from the center by 1 to 5 aa or 1 to 10 aa and an adjacent amino acid.

The ABM may, for example, be an albumin protein domain, affibody, or peptide. The albumin protein domain, affibody, or peptide is small in size compared to antibodies (IgG) and is fused to the scFv to construct a single polypeptide chain. The albumin protein domain, affibody, or peptide may be a disulfide bond-free structure.

The ABM may be a part of a specific protein or may be an artificially designed domain.

For example, the ABM may be a small triple helix protein domain found in various surface proteins expressed by Gram-positive bacteria. Specifically, the ABM may be derived from streptococcal protein G or protein PAB (Peptostreptococcal albumin-binding protein) of Finegoldia magna or may be those modified to further increase albumin-binding affinity.

For example, the ABM may be a VNAR (V domain of the cartilage oligomeric matrix protein, chondrocyte-derived) derived from sea sharks, or may be those modified to further increase albumin-binding affinity.

Specifically, the ABM may have an amino acid sequence of SEQ ID NOs: 1 to 3.

The modified protein of the present disclosure may be prepared in a microorganism in which a sequence encoding this protein is expressed.

As the microorganism, any microorganism known in the art may be used without limitations. For example, the microorganism may be Escherichia coli, but there are no limitations.

In the case of preparing the modified protein using a microbial system, the expression is facilitated only when the protein does not involve post-translational modifications (PTMs) and contains none or fewer disulfide bonds. Antibody forms, such as IgG, contain many disulfide bonds and involve PTMs, and additional coupling and purification processes are required when using compounds. The modified protein of the present disclosure is characterized by being in the form of a fused polypeptide, not involving PTMs, and containing fewer disulfide bonds, and thus can facilitate the microbial system-based expression.

In the preparation process, the sequence of the linker in the above-described scFv may be partially replaced with, or further comprise, sites recognizable by restriction enzymes so that the ABM is inserted during gene cloning. For example, the sequence of the linker, flanking both sides of the ABM, may be partially replaced with, or further comprise, a TS sequence, which is a site recognizable by the restriction enzyme (Spel). The residue in the TS sequence has benefits similar to those of the residue in the GS sequence of the linker.

The present disclosure provides a pharmaceutical composition for the prevention or treatment of cancer, including the protein above. All the details described in terms of the protein above are applied exactly to the protein serving as an active ingredient in the pharmaceutical composition of the present application.

This cancer may be selected from the group consisting of, for example, brain cancer, head and neck cancer, bladder cancer, breast cancer, cervical cancer, colon cancer, colorectal cancer, endometrial cancer, esophageal cancer, leukemia, lung cancer, liver cancer, ovarian cancer, pancreatic cancer, prostate cancer, rectal cancer, kidney cancer, stomach cancer, testicular cancer, uterine cancer, vascular tumor, squamous cell carcinoma, adenocarcinoma, small-cell carcinoma, melanoma, glioma, neuroblastoma, sarcoma, laryngeal cancer, parotid gland cancer, biliary tract cancer, thyroid cancer, actinic keratosis, acute lymphoblastic leukemia, acute myeloid leukemia, adenoid cystic carcinoma, adenoma, adenosquamous carcinoma, anal canal cancer, anal cancer, anorectal cancer, astrocytoma, Bartholin gland cancer, basal cell carcinoma, biliary cancer, bone cancer, bone marrow cancer, bronchial cancer, bronchial adenocarcinoma, carcinoid, cholangiocarcinoma, chronic lymphoblastic leukemia, chronic myeloid leukemia, clear cell carcinoma, connective tissue cancer, cystadenoma, digestive system cancer, duodenal cancer, endocrine system cancer, endodermal sinus tumor, endometrial hyperplasia, endometrioid adenocarcinoma, endothelial cell cancer, ependymal cells, epithelial cell cancer, orbital cancer, focal nodular hyperplasia, gallbladder cancer, pyloric cancer, gastric fundus cancer, gastrinoma, glioblastoma, glucagonoma, heart cancer, hemangioblastoma, hemangioendothelioma, hemangioma, hepatic adenoma, hepatic adenomatosis, hepatobiliary cancer, hepatocellular carcinoma, Hodgkin's disease, ileal cancer, insulinoma, intraepithelial neoplasia, intraepithelial squamous cell neoplasia, intrahepatic biliary cancer, invasive squamous cell carcinoma, jejunal cancer, joint cancer, pelvic cancer, giant cell carcinoma, large intestine cancer, lymphoma, malignant mesothelial cell tumor, medulloblastoma, medulloepithelioma, meningeal cancer, mesothelial cancer, metastatic carcinoma, oral cancer, mucoepidermoid carcinoma, multiple myeloma, muscle cancer, nasal cavity cancer, nervous system cancer, non-epithelial skin cancer, non-Hodgkin's lymphoma, oat cell carcinoma, oligodendroglioma cancer, oral cancer, osteosarcoma, papillary serous adenocarcinoma, penile cancer, pharyngeal cancer, pituitary tumor, plasmacytoma, pseudosarcoma, pulmonary blastoma, rectal cancer, renal cell carcinoma, respiratory system cancer, retinoblastoma, serous carcinoma, sinus cancer, skin cancer, small-cell carcinoma, small intestine cancer, leiomyosarcoma, soft tissue cancer, somatostatin-secreting tumor, spinal cancer, squamous cell carcinoma, striated muscle cancer, submesothelial cell cancer, T cell leukemia, tongue cancer, ureteral cancer, urethral cancer, cervical cancer, corpus cancer, vaginal cancer, VIPoma, vulvar cancer, well-differentiated carcinoma, and Wilms tumor.

The pharmaceutical composition of the present disclosure may include a pharmaceutically acceptable carrier. As used herein, the term “pharmaceutically acceptable carrier” refers to a carrier or diluent that does not cause significant irritation in organisms without compromising the biological activity and properties of the administered components. As the pharmaceutically acceptable carrier of the present disclosure, saline, sterile water, Ringer's solution, buffered saline, dextrose solution, maltodextrin solution, glycerol, ethanol, or a mixture of one or more components of the foregoing may be used. In addition, the pharmaceutically acceptable carrier may be formulated as injectable forms suitable for injection into the tissues or organs by adding other common additives, such as antioxidants, buffers, and bacteriostats, as needed. Furthermore, the pharmaceutically acceptable carrier may be formulated as isotonic sterile solutions or, in some cases, as dry preparations (especially lyophilized preparations) that can be made into injectable solutions by adding sterile water or normal saline. To function specifically on target organs, target organ-specific antibodies or other ligands may also be bound to the carrier for use.

In addition, the composition of the present disclosure may further include a filler, an excipient, a disintegrant, a binder, or a lubricant. Furthermore, the composition of the present disclosure may be formulated using methods known in the art to provide rapid, sustained, or delayed release of the active ingredient after administration to a mammal.

In one embodiment, the pharmaceutical composition may be an injectable formulation and administered intravenously, but there are no limitations.

As used herein, the term “effective amount” refers to an amount required to delay or entirely promote the onset or progression of a particular disease targeted for treatment.

In the present disclosure, a pharmaceutically effective amount of the composition may be administered. It will be apparent to those skilled in the art that the appropriate total daily dosage of the pharmaceutical composition can be determined by prescribing physicians within a proper range of medical judgment.

For the purposes of the present disclosure, the specific pharmaceutically effective amount for a particular patient is preferably applied differently depending on the type and degree of response to be achieved, specific compositions, including whether other preparations are used in some cases, various factors such as the age, weight, general health status, sex, and diet of the patient, administration time, administration route, the secretion rate of the composition, treatment duration, and drugs used with or concurrently with the specific compositions, and similar factors well known in the medicine fields.

In the present disclosure, the pharmaceutical composition may be accompanied, as needed, by an instruction associated with the packaging in a form directed by a governmental agency in charge of the preparation, use, and sale of drugs. In addition, the instruction indicates approval by a private agency in terms of the form of the composition or administration to humans or animals and may, for example, be a label approved by the U.S. FDA for the prescription of a drug.

The present disclosure provides a method of treating cancer, including a step of administering the protein above. All the details described in terms of the protein above are applied exactly to the protein serving as an active ingredient in the method of treating cancer of the present application.

The method of the present disclosure comprises a step of administering the protein to a subject having cancer.

The subject having cancer may be an animal having cancer, which may be, specifically, a mammal having cancer and, more specifically, a human having cancer.

A therapeutically effective amount of the protein may be administered.

As used herein, the term “administration” refers to introducing the composition of the present disclosure into a patient by any suitable method. In terms of administration route, the composition of the present disclosure may be administered via various oral or parenteral routes as long as the composition can reach a target tissue. The composition of the present disclosure may be administered intraperitoneally, intravenously, intramuscularly, subcutaneously, intradermally, orally, topically, intranasally, intrapulmonarily, or intrarectally, but there are no limitations.

Hereinafter, the present disclosure will be described in more detail with reference to examples.

### Example

Examples 1 to 9 and Comparative Examples 1 to 4 were designed by varying scFvs, the type of ABD, and the location of the ABD, followed by identifying the structural models thereof. The amino acid sequence of the scFv protein is composed of an N-terminal V_{H} domain and a C-terminal V_{L} domain linked by a linker. An “MG” sequence was inserted at the N-terminal as a restriction site for the NcoI enzyme, and a hexahistidine tag (His-tag) sequence was inserted at the C-terminal as a protein purification tag. The Spel restriction site encoding the amino acid residue “TS” was used for ABD insertion.

TABLE 1

| | | SEQ | |
|---|---|---|---|
| | | ID | FIG. |
| Category | Label | NO. | No. |
| Example 1 | 4D5-S-ABD | 13 | 2 |
| Example 2 | 4D5-L-ABD | 14 | 3 |
| Example 3 | 4D5-LinK_{N}-ABD | 15 | 4 |
| Example 4 | 4D5-LinK_{c}-ABD | 16 | 5 |
| Example 5 | 4D5-Term_{N}-ABD | 17 | 6 |
| Example 6 | OKT3-ABD | 18 | 7 |
| Example 7 | 3F8-ABD | 19 | 8 |
| Example 8 | 3F8-VNAR | 20 | 9 |
| Example 9 | 3F8-ABP(Cyc Y12H) | 21 | 10 |
| Comparative example 1 | 4D5-V_{H}-ABD(loop region) | 22 | 11 |
| Comparative example 2 | 4D5-V_{H}-ABD(beta sheet region) | 23 | 12 |
| Comparative example 3 | 4D5-V_{L}-ABD(loop region) | 24 | 13 |
| Comparative example 4 | 4D5-V_{L}-ABD(beta sheet region) | 25 | 14 |

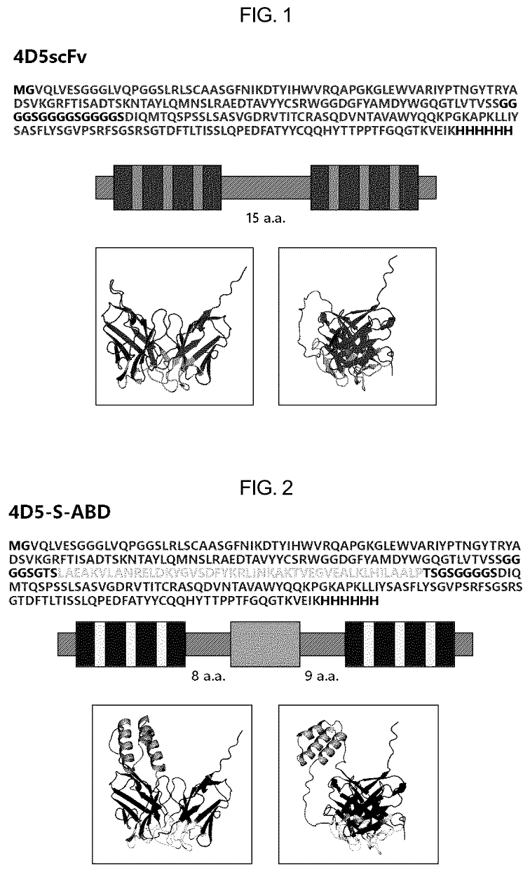

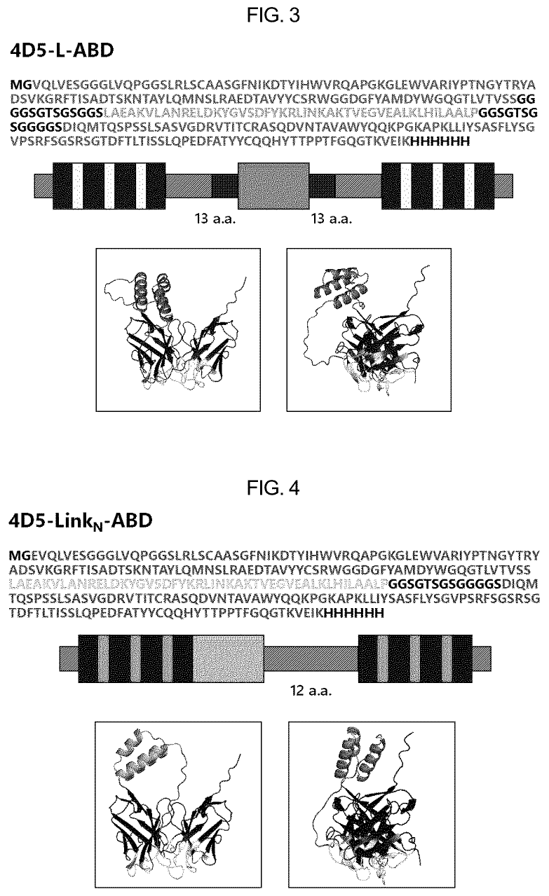

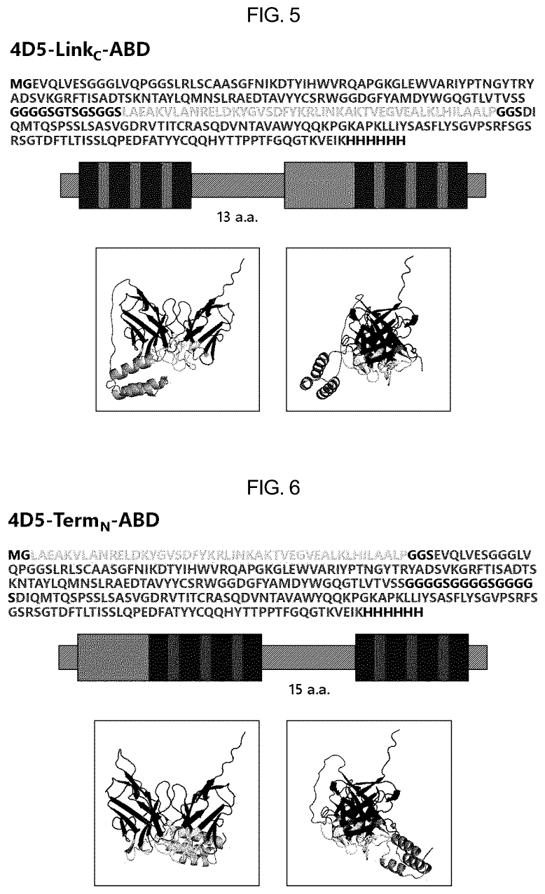

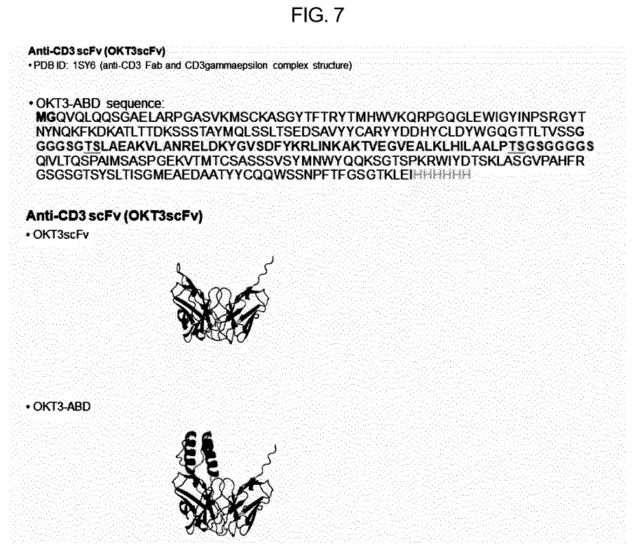

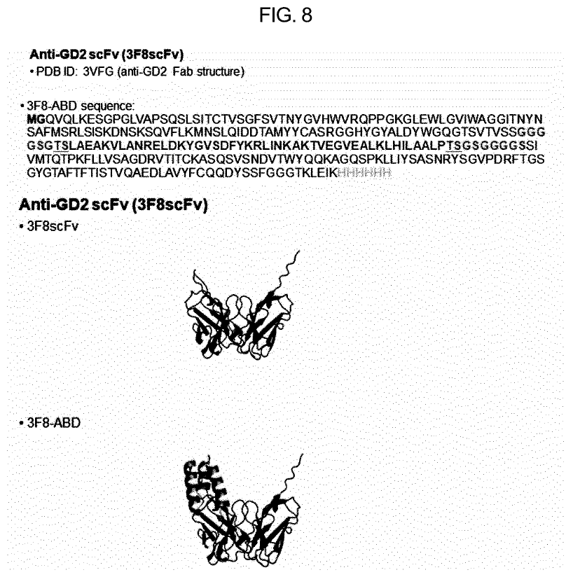

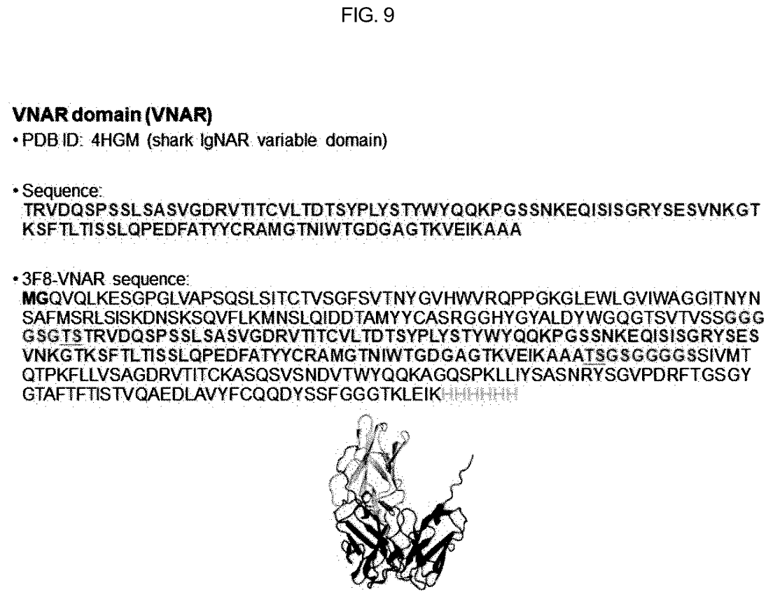

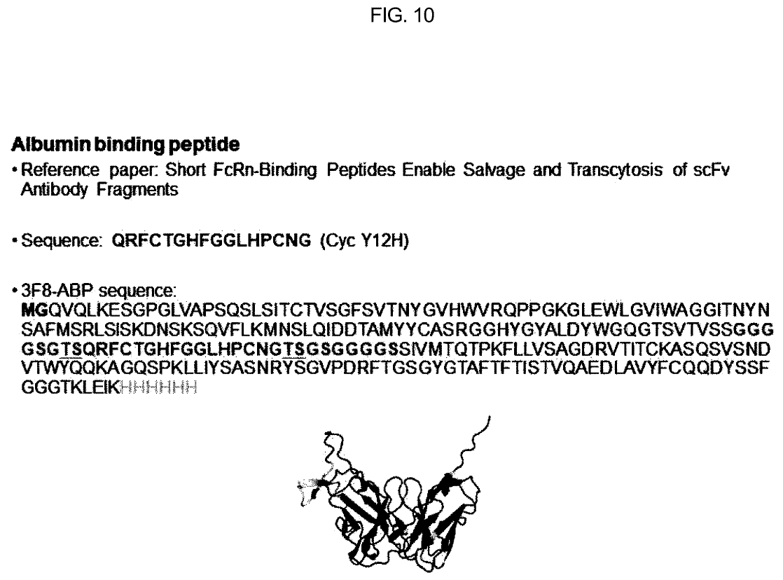

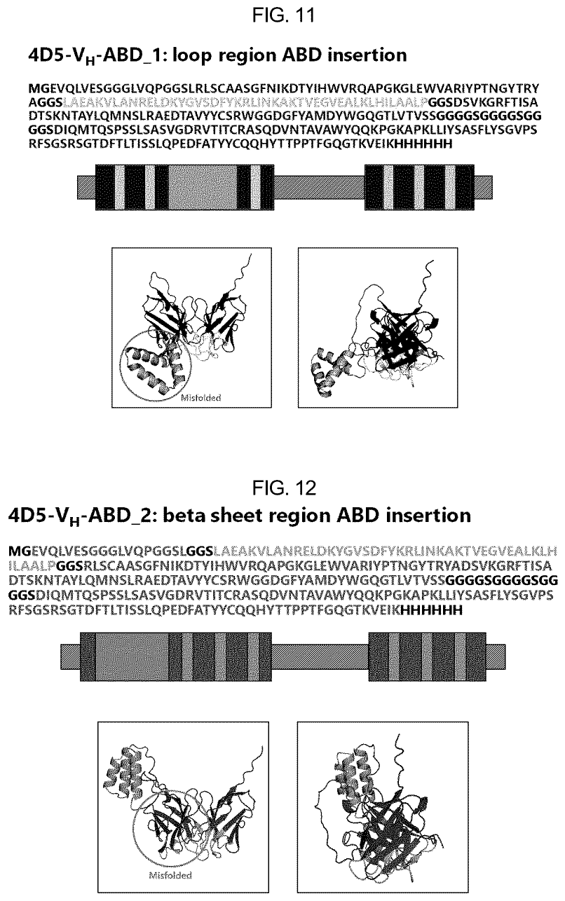

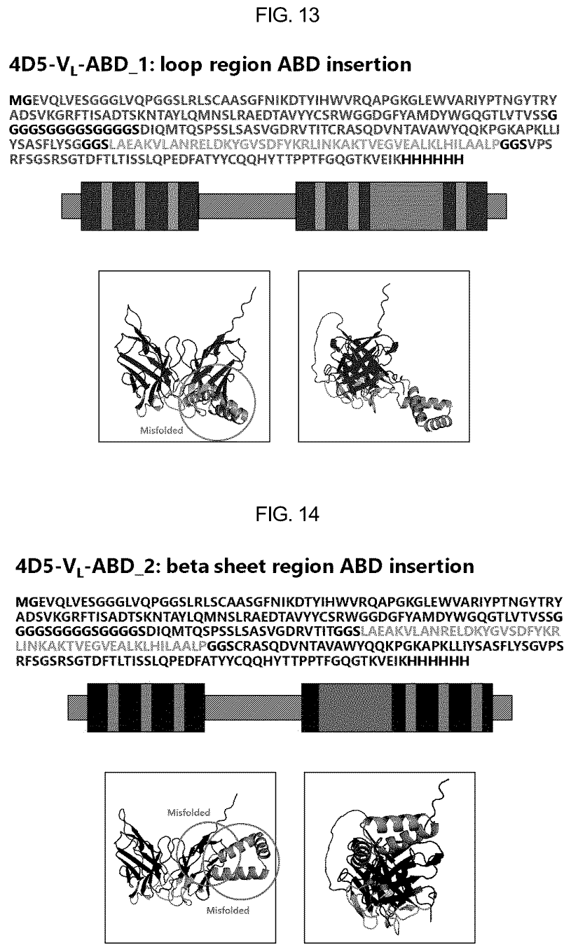

FIGS. 1 to 14 illustrates the structures of 4D5 scFv (SEQ ID NO: 4, FIG. 1), Examples 1 to 9, and Comparative Examples 1 to 4, calculated using AlphaFold2. In the drawings, the three bright regions within the respective heavy chain and light chain variable domain indicate CDRs. All examples confirmed that the protein structures were fused within the linker and folded ordinarily without compromising the structure formed of the heavy chain and light chain variable domain. FIG. 1 illustrates the structure of the original ABD-free 4D5scFv, which was implemented by AlphaFold2 using the ColabFold web server. In comparison, the original structure of 4D5Ab was retained substantially in Examples 1 to 5, confirming that anti-HER2 binding affinity would be maintained.

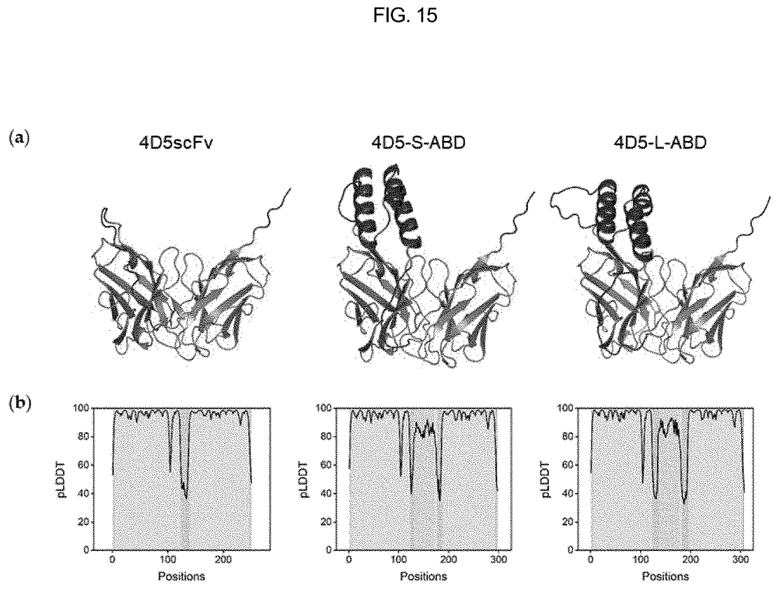

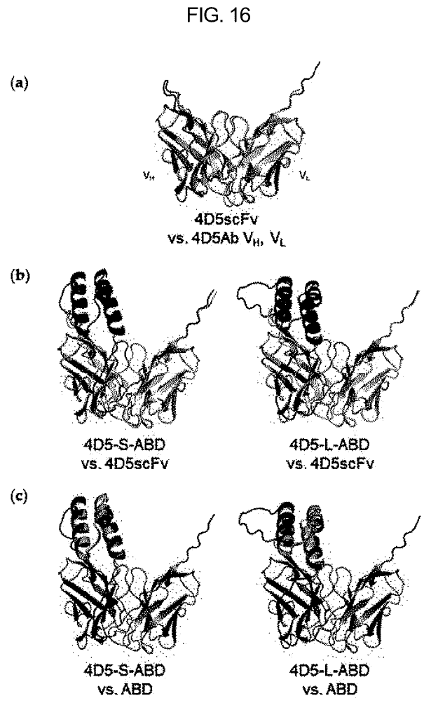

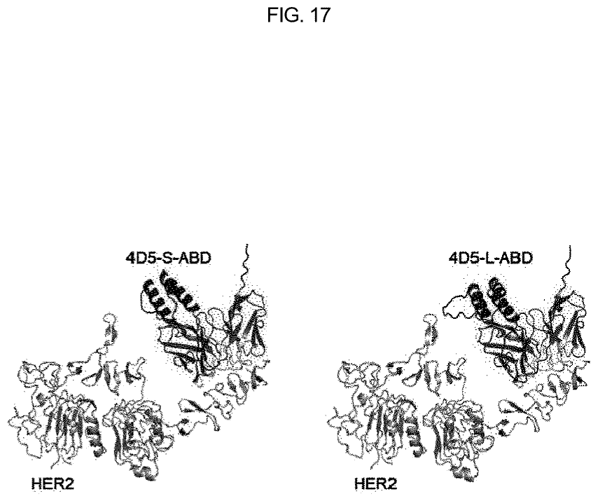

In addition, 4D5-S-ABD and 4D5-L-ABD were analyzed by pTM scores and pLDDT. These structures were computationally predicted using AlphaFold2 and analyzed through pTM scores and pLDDT to identify the structural integrity of those 4D5-ABD variants in such unfamiliar forms (FIG. 15, Table 2). Then, the model structures of the 4D5-ABD variants were aligned with those of 4D5scFv and the ABD (FIG. 16, Table 3). The TM scores for all alignments were 0.94 or higher, which was significantly high. In other words, the 4D5scFv and ABD structures are retained substantially in the 4D5-ABD variants constructed, meaning that the functions thereof would also be maintained. In addition, the predicted model structure showed that the ABD insertion location was distant from the CDR of 4D5scFv, as expected. This fact is also observable in the visualization of 4D5-ABD and HER2 complexes (FIG. 17), in which case the ABD is clearly separated from the HER2 binding site. Thus, there may be a prediction that the antigen-binding affinity of 4D5scFv will not be significantly hindered by the presence of the ABD or the binding of albumin to the ABD. In conclusion, the high structural consistency and locational independence of each component allow 4D5scFv and the ABD to substantially retain the functions thereof.

TABLE 2

| Score | 4D5scFv | 4D5-S-ABD | 4D5-L-ABD |
|---|---|---|---|
| pTM score | 0.88 | 0.76 | 0.75 |
| Mean pLDDT (whole) | 91.7 | 89.6 | 88.7 |
| Mean pLDDT (V_{H}/V_{L} only) | 95.6 | 94.7 | 95.3 |
| Mean pLDDT (ABD only) | — | 83.1 | 84.8 |

TABLE 3

| Score | 4D5scFv |
|---|---|
| 4D5scFv vs. 4D5Ab V_{H} | 0.98 |
| 4D5scFv vs. 4D5Ab V_{L} | 0.99 |
| 4D5-S-ABD vs. 4D5scFv | 0.97 |
| 4D5-L-ABD vs. 4D5scFv | 0.95 |
| 4D5-S-ABD vs. ABD | 0.95 |
| 4D5-L-ABD vs. ABD | 0.94 |

### Experimental Example

### 1. Expression and Purification of Protein

### 1.1. Plasmid

Among the modified proteins constructed by the above design, those of Examples 1 to 5 were subjected to cytoplasmic expression in a bacterial host to produce proteins. Specifically, the gene encoding the above amino acid sequence was optimized using the ExpOptimizer (NovoPro Bioscience, Shanghai, China), synthesized and subcloned by Macrogen (Seoul, Korea), and inserted into the NcoI/KpnI restriction site in the pBAD vector, thereby producing pBAD_4D5scFv plasmids.

The plasmids of Examples 1 to 5 and 4D5scFv were transformed into Escherichia coli TOP10 host cells. The transformed cells were incubated in a standard 2×YT medium containing 100 μg/mL ampicillin for 16 hours at 37° C. while being shaken at 200 rpm. These cells were inoculated into the same fresh medium and incubated until the optical density thereof reached 0.5 to 0.6 at 600 nm (OD600). Protein expression was induced by L-(+)-arabinose at a final concentration of 0.2% (w/v). After cooling the temperature to 23° C., the culture solution was incubated for 24 hours, and the grown cells were centrifuged at 8000 rpm at 4° C. and harvested. Then, the cell pellets were stored at −80° C. Samples for each variant before induction (BI) and after induction (AI) were collected, centrifuged at 13,000 rpm for 1 minute, and resuspended in PBS (pH 7.4) containing 2 M urea, thereby performing SDS-PAGE analysis.

The above His-tagged examples were purified by metal affinity chromatography using Ni-NTA. The cell pellets were lysed with 1 mg/mL lysozyme and 5 mg/mL Dnase in a lysis buffer (10 mM imidazole, 50 mM NaH₂PO₄, 300 mM NaCl, pH 8.0) and incubated on ice for at least 5 minutes. The resulting solution was sonicated for a total of 15 minutes with a 1-second pulse and a 2-second rest at 28% amplitude (500 W, 20 kHz). After a 5-minute rest, this process was repeated. Thereafter, the resulting mixture was centrifuged at 10,000×g for 20 minutes at 4° C., and the supernatant was incubated with a Ni-NTA agarose resin for 30 minutes at 4° C. The incubated resin was loaded onto a polypropylene column with a filter, washed with a wash buffer (20 mM imidazole, 50 mM NaH₂PO₄, 300 mM NaCl, pH 8.0), and eluted with an elution buffer (250 mM imidazole, 50 mM NaH₂PO₄, 300 mM NaCl, pH 8.0). The buffer was exchanged with PBS (pH 7.4) using a PD-10 desalting column according to the manufacturer's protocol. The purified proteins were stored at 4° C. and analyzed by SDS-PAGE.

### 1.2. SDS-PAGE Analysis

Such prepared proteins, including the resuspended BI and Al samples and the purified proteins, were analyzed using SDS-PAGE. All samples were mixed with a 2×sample loading dye containing 100 mM dithiothreitol (DTT) (0.2% bromophenol blue, 4% SDS, 20% glycerol, 100 mM Tris-HCl, pH 6.8) for reduction. DTT was omitted to prepare non-reducing samples. The samples were boiled at 100° C. for 10 minutes and then introduced into a 12% SDS-PAGE gel. After the electrophoresis, the gel was stained with Coomassie brilliant blue (0.25% Coomassie blue, 50% ethanol, 10% acetic acid) and destained (50% ethanol, 10% acetic acid). The resulting gel was imaged and visualized using the ChemiDoc XRS+ system (Bio-Rad, Hercules, CA, USA).

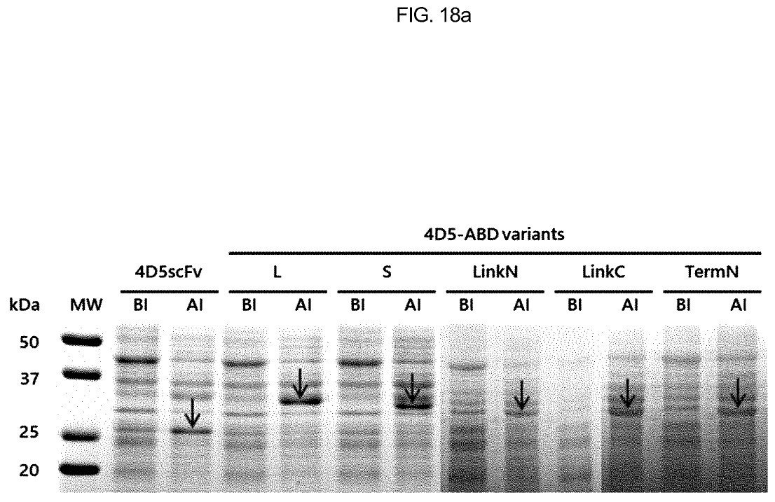

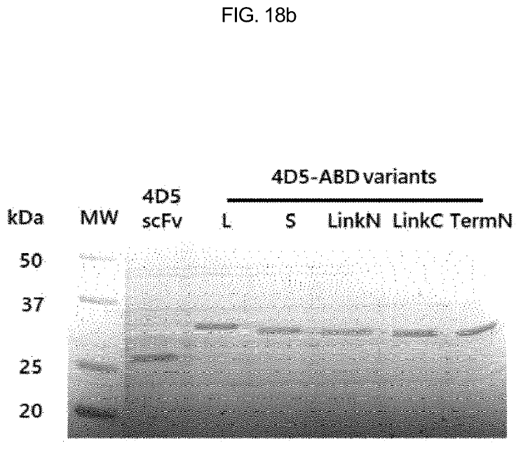

FIG. 18a shows the SDS-PAGE analysis results of the cell lysates collected during the expression. Bands were detected in the lanes loaded with the Al samples having molecular weights of 27, 32, and 33 kDa, respectively, which correspond to the molecular weights of 4D5scFv, 4D5-S-ABD, and 4D5-L-ABD. The purification was performed using the interaction between the Ni-NTA in the agarose resin and the His-tag affinity tag at the C-terminal of each protein. As a result of the SDS-PAGE analysis of the purified proteins, bands at the same position as in the expression gels were detected (FIG. 18b).

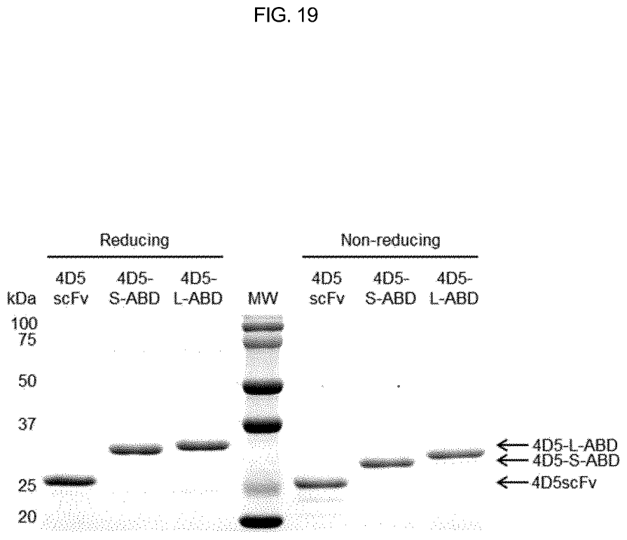

In addition, as a result confirmed through Examples 1 and 2, only monomeric protein bands were shown under both the reducing and non-reducing conditions of the modified proteins (FIG. 19), meaning that there were no substantial intermolecular disulfide bonds formed.

### 1.3. MALDI-TOF Analysis

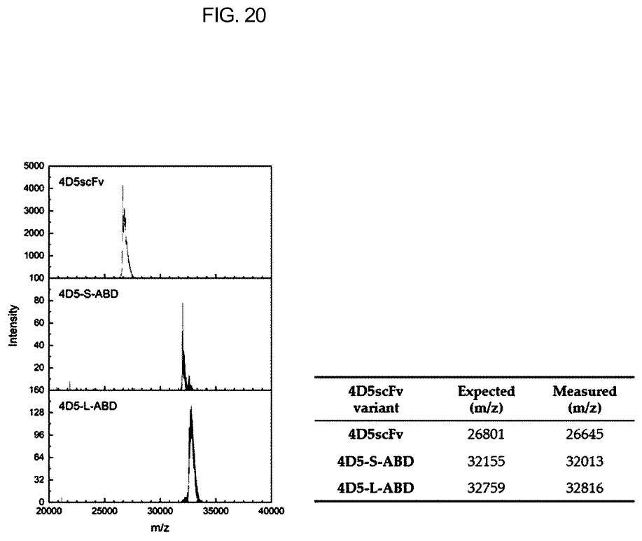

MALDI-TOF analysis was performed. Each of the purified samples was desalted using a Zeba spin desalting column (7K MWCO) in PBS (pH 7.4) and prepared with a 0.1% TFA solution. The sample was mixed at a 1:1 (v/v) ratio with a SA matrix saturated in a TA30 (30% acetonitrile, 0.1% TFA) solution. The SA matrix saturated in an ethanol solution was pre-deposited on a steel target plate. Then, the solution in which the sample was mixed was deposited on top of the layer to perform molecular weight analysis using the Autoflex Speed (Bruker, Billerica, MA, USA). Protein Standard II was used as the calibration standard. These three proteins were identified additionally by MALDI-TOF analysis (FIG. 20).

In FIG. 20, peaks at 26,645, 32,013, and 32,816 m/z were detected in the mass spectra of intact 4D5scFv, 4D5-S-ABD, and 4D5-L-ABD. These results aligned in a deviation of less than 1% well with the expected values of the 4D5scFv variants at 26,801, 32,155, and 32,759 m/z, respectively. The production yields of purified 4D5scFv, 4D5-L-ABD, and 4D5-S-ABD were 22.0±0.5, 18.8±1.3, and 21.5±0.8 mg/L, respectively. Overall, these results show that the 4D5scFv variant samples were successfully expressed and purified.

### 1.4. Size Exclusion Chromatography (SEC) Analysis

Additionally, the purified samples of Examples 1 and 2 were characterized by Fast Protein Liquid Chromatography SEC(Size Exclusion Chromatography). Equimolar (5 μM) samples prepared in PBS (pH 7.4) were injected into a Superdex 75 Increase 10/300 GL column (Cytiva, Uppsala, Sweden) and eluted at a flow rate of 0.2 mL/min. Ovalbumin, Conalbumin, and Aldolase from the gel filtration calibration kit HMW (Cytiva, Uppsala, Sweden) were used as standard proteins. The analysis was performed using a chromatography system, NGC Quest 10 (Bio-Rad, Hercules, CA, USA).

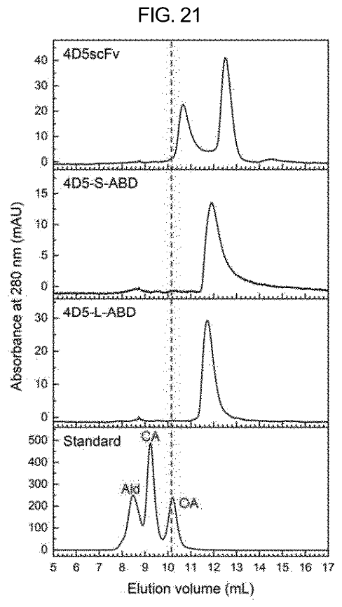

The SEC analysis results in FIG. 21 showed that 4D5scFv was stable in the mixture of monomers and dimers, and there were no disulfide bonds formed according to non-reducing SDS-PAGE. In contrast, the 4D5-ABD variants did not form dimers but existed only as monomers, which may be due to the steric hindrance created by the ABD fusion.

### 2. Binding Affinity Analysis

### (1) Binding Affinity Analysis to HER2

The anti-HER2 targeting efficiency of the purified 4D5scFv variants was analyzed through anti-HER2 ELISA in the presence or absence of HSA. As a specific method, an immunoplate was coated with a recombinant HER2 antigen (500 pg/μL, 100 μL/well), which had been incubated overnight at 4° C. in a coating buffer (PBS, pH 7.4). This plate was shaken with PBS with 0.05% Tween 20 (PBST) at 200 μL/well and washed three times. Then, the plate was blocked by introducing a blocking buffer (5% skimmed milk) in PBST for room-temperature incubation at 200 μL/well and then washed four times. The purified protein was incubated at room temperature in PBS (pH 7.4) with or without HSA at a 1:2 molar ratio. Then, the incubated protein was prepared in the blocking buffer to make a 50 nM stock, which was subjected to 3-fold dilution in succession. The prepared sample (100 μL/well, n=2) was loaded onto the plate and incubated at room temperature, followed by washing unbound proteins four times. The plate was incubated with 100 μL/well of rabbit anti-His-tagged antibodies diluted to 1:1500 in the blocking buffer. After the incubation, each well was washed four times. Lastly, 100 μL/well of HRP-binding anti-rabbit IgG antibodies diluted to 1:3000 was incubated together in the blocking buffer. Each well was washed four times. After performing detection by adding 100 μL/well of TMB, the reaction was quenched with 2 M HCl. The absorbance of the bound proteins was measured at 450 nm for quantification.

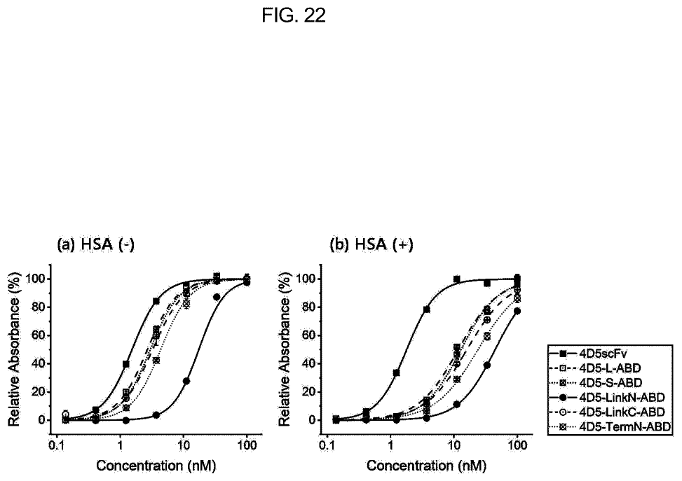

The experimental results are shown in FIG. 22 and Table 4 below. All proteins showed similar concentration-dependent curves, meaning that the ABD insertion did not cause significant distortion in the 4D5scFv structure. In conclusion, this suggests that the anti-HER2 binding affinity of the 4D5-ABD variants is maintained.

Specifically, for the comparison of the binding capacity when albumin is binding to the ABD or not, experimental groups (HSA+), in which antibodies and HSA were pre-incubated, and experimental groups (HSA−), in which antibodies and HSA were not incubated, were compared. The HER2 binding of ABD-free 4D5scFv does not change significantly, indicating that the anti-HER2 binding affinity of 4D5scFv is not hindered or blocked by the presence of HSA. When HSA binds to the 4D5-ABD variants, the anti-HER2 binding affinity may slightly decrease. This may be because when bulky HSA binds to 4D5scFv, the steric hindrance to HER2 is weak. Both in the presence and absence of HSA, 4D5-L-ABD and 4D5-S-ABD with the ABD positioned in the center of the linker showed relatively strong binding capacity, among which 4D5-L-ABD with the longest linker length showed the strongest binding capacity. LinkC and TermN, expected to have low binding affinity because the ABD is positioned near the CDR on the basis of the structure predicted by AlphaFold2, were unexpectedly shown to have higher binding affinity than the LinkN variant in which the ABD is distant from the CDR. This is presumed to be because linker residues are inserted at a minimum at the ABD C-term in the case of LinkC and TermC, whereas LinkN is free of additional linker residues at the N-term. Such differences occur during the cloning process to create each variant. This means that steric hindrance occurs when there is fewer or no linker between the ABD and the antibody, and the linker is required at a minimum between the antibody and the ABD for binding capacity. Thus, it is seen that 4D5-S-ABD and 4D5-L-ABD, in which the antibody and the ABD are affected by steric hindrance the least by inserting the ABD into the center of the linker region, have the highest binding capacity.

TABLE 4

EC₅₀ (nM)

| Sample | HSA (−) | HSA (+) |
|---|---|---|
| 4D5scFv | 1.54 ± 0.03 | 1.79 ± 0.11 |
| 4D5-L-ABD | 2.68 ± 0.08 | 11.72 ± 1.81 |
| 4D5-S-ABD | 2.98 ± 0.11 | 12.88 ± 1.25 |
| 4D5-LinkN-ABD | 17.39 ± 0.34 | 44.61 ± 3.01 |
| 4D5-LinkC-ABD | 3.24 ± 0.14 | 15.99 ± 1.45 |
| 4D5-TermN-ABD | 4.35 ± 0.06 | 23.93 ± 2.70 |

### (2) HSA Binding Affinity Analysis

The anti-HSA efficiency of the purified 4D5-ABD variants was also investigated by ELISA. As a specific method, a plate was coated with HSA (500 pg/μL) in a coating buffer. The purified protein was prepared at 20 nM in a blocking buffer and then subjected to 3-fold dilution in succession. All other conditions and procedures were performed as described above.

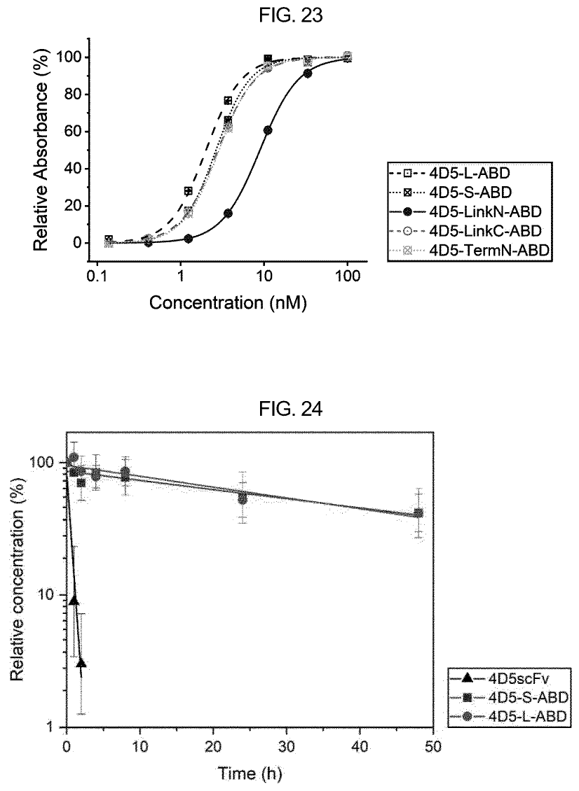

To investigate the binding affinity of the 4D5scFv variants in the presence of HSA, ELISA was performed after pre-incubating all variants with HSA at a 1:2 molar ratio. The results of the anti-HSA binding affinity are shown in FIG. 23 and Table 5 below.

4D5-L-ABD and 4D5-S-ABD were shown to have the strongest binding capacity. The binding capacity of the ABD tends to decrease slightly and thus appears to be affected less by linker length than that of the antibody, but the tendency was the same as that of the antibody.

TABLE 5

| Sample | EC₅₀ (nM) |
|---|---|
| 4D5scFv | n.d. |
| 4D5-L-ABD | 2.01 ± 0.11 |
| 4D5-S-ABD | 2.65 ± 0.15 |
| 4D5-LinkN-ABD | 8.91 ± 0.18 |
| 4D5-LinkC-ABD | 2.80 ± 0.06 |
| 4D5-TermN-ABD | 2.88 ± 0.09 |

### 3. Pharmacokinetic Analysis

For pharmacokinetic analysis, the serum half-lives of the 4D5scFv variants were investigated using mice. The experiments were performed in accordance with the guidelines of the Institutional Animal Care and Use Committee of the Gwangju Institute of Science and Technology (GIST). Specifically, 4D5scFv and the 4D5-ABD variants (100 μg/mL) purified in 200 μL PBS (pH 7.4) were injected into the tail vein of 9-week-old female BALB/c mice (n=4). Blood samples (<100 μL) were collected 3 minutes, 1, 2, 4, 8, 24, and 48 hours after the injection through retro-orbital blood collection. The serum was centrifuged at 10,000×g for 10 minutes at 4° C., separated from the blood, and then stored at −20° C. until further use. The serum protein concentrations at each time point were measured by ELISA, as described above. The concentration was calculated by interpolating a standard calibration curve.

The serum half-lives of 4D5scFv and the 4D5-ABD variants were measured after the intravenous injection into the BALB/c mice (n=4). ELISA was performed to quantify the protein remaining in the serum (FIG. 24). While 4D5scFv was rapidly removed from the blood with a serum half-life of 18 minutes, 4D5-S-ABD and 4D5-L-ABD showed a significantly extended half-life of 34 hours, which is a value greater than that of 4D5scFv by 114 times. In addition, the values of AUC were increased by about 70 times in the case of both ABD fusion variants and calculated as 42, 2887, and 3008 in the case of 4D5scFv, 4D5-S-ABD, and 4D5-L-ABD, respectively. Considering that the 4D5-ABD variants did not reach the detection limit, the actual AUC increase is expected to be much higher.

TABLE 6

| 4D5scFv Variant | t_{1/2} (h) | AUC (% · h) |
|---|---|---|
| 4D5scFv | 0.30 ± 0.25 | 42 ¹ |
| 4D5-S-ABD | 34.19 ± 0.05 | 2887 ² |
| 4D5-L-ABD | 34.29 ± 0.05 | 3008 ² |

In the above,
¹ 0 to 2 h;
² 0 to 48 h.

The significant increases in the half-lives of the 4D5-ABD variants may be inferred to be because the 4D5-ABD molecules binding to albumin were subjected to FcRn-mediated recycling. This result also shows that the internally inserted ABD allows the binding affinity to serum albumin to be maintained, corresponding to the structural calculations and the ELISA results. In addition, no difference was observed between the 4D5-ABD variants, also corresponding to the ELISA results. This also suggests that the linker length of 15 amino acid residues in the 4D5-S-ABD variants is sufficient to allow the ABD moiety to move freely for binding to albumin.

1.-14. (canceled)

15. A modified protein comprising: 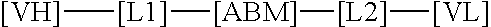
wherein,
the VH is a heavy chain variable domain;
the L1 is a first linker comprising at least 3 amino acids;
the ABM is an albumin binding moiety which is capable of binding to an albumin;
the L2 is a second linker comprising at least 3 amino acids; and
the VL is a light chain variable domain,
wherein the VH together with the VL are capable of binding to a specific antigen, and
wherein the albumin binding moiety is a polypeptide.

16. The modified protein of claim 15, wherein the albumin binding moiety has no disulfide bond, and
wherein the modified protein has no post-translational modification (PTM).

17. The modified protein of claim 15, wherein the length of the albumin binding moiety is 10 to 150 aa.

18. The modified protein of claim 15, wherein the first linker and the seconds linker comprise more than 8 amino acids, respectively.

19. The modified protein of claim 15, wherein the length of the VH and the VL is 70 to 150 aa, respectively.

20. The modified protein of claim 15, wherein the albumin binding moiety comprises an amino acid sequence selected from a sequence of SEQ ID NO. 1 to 3.

21. The modified protein of claim 15, wherein the first linker and the second linker comprise an amino acid sequence of (GS)n, (GGS)n (GGGGS)n or a combination thereof, respectively, and
wherein the n is a natural number selected from 1 to 10, respectively.

22. The modified protein of claim 15, wherein the VH and the VL are derived from same antibody, and
wherein the antibody is selected from the group consisting of trastuzumab, naxitamab, cetuximab, telisotuzumab, tisotumab, pinatuzumab, lifastuzumab, indusatumab, vandortuzumab, sofituzumab, vorsetuzumab, trastuzumab, mirvetuximab, coltuximab, naratuximab, indatuximab, anetumab, lorvotuzumab, cantuzumab, laprituximab, bivatuzumab, vadastuximab, rovalpituzumab, inotuzumab, sacituzumab, labetuzumab, milatuzumab, lupartumab, aprutumab, pembrolizumab, atezolizumab, and OKT3 (Anti-CD3 monoclonal antibody).

23. The modified protein of claim 15, wherein a bioactive substance is fused at the C-terminal of the modified protein.

24. The modified protein of claim 23, wherein the bioactive substance is selected from the group consisting of cytokine, insulin, insulin-like growth factor (IGF)-1, IGF-2, epidermal growth factor (EGF), transforming growth factor (TGF), human growth hormone, and vascular endothelial growth factor (VEGF).
